(19) United States
(12) Patent Application Publication
(10) Pub. No.: US 2021/0045731 A1
(43) Pub. Date: Feb. 18, 2021

(54) TRANS-RADIAL CLOSURE DEVICE, DEPLOYMENT APPARATUS, AND METHOD OF DEPLOYING A TRANS-RADIAL CLOSURE DEVICE

(71) Applicant: TRANSLUMINAL TECHNOLOGIES, LLC, SYRACUSE, NY (US)

(72) Inventors: Stephen M. Green, Syracuse, NY (US); Ronald P. Caputo, Manlius, NY (US); John M. Kirwan, Wibraham, MA (US)

(73) Assignee: TRANSLUMINAL TECHNOLOGIES, LLC, SYRACUSE, NY (US)

(21) Appl. No.: 16/966,537

(22) PCT Filed: Feb. 1, 2019
(86) PCT No.: PCT/US19/16359

§ 371 Date: Jul. 31, 2020

Related U.S. Application Data
(60) Provisional application No. 62/624,854, filed on Feb. 1, 2018.

(51) Int. Cl. A61B 17/04; A61B 90/00

(52) U.S. Cl. CPC A61B 17/0401; A61B 90/39; A61B 2017/00367; A61B 2017/0409; A61B 2017/0406

(57) ABSTRACT

The present invention provides an apparatus and method for creating hemostasis at a subcutaneous vascular puncture. The method and apparatus is intended, but not limited to, vascular punctures following trans-radial arterial procedures, e.g. catheterization and percutaneous coronary intervention.

### CROSS-REFERENCE TO RELATED APPLICATIONS

This application claims priority to U.S. Provisional Patent Application Ser. No. 62/624854, filed on Feb. 1, 2018 and entitled “TRANS-RADIAL CLOSURE DEVICE, DEPLOYMENT APPARATUS, AND METHOD OF DEPLOYING A TRANS-RADIAL CLOSURE DEVICE”

### FIELD OF THE INVENTION

The present invention relates to medical devices, and more particularly, to a vascular puncture hemostasis apparatus following trans-radial arterial procedures.

### BACKGROUND OF THE INVENTION

Various medical procedures, particularly cardiology procedures, involve accessing a corporeal blood vessel through a percutaneous sheath. Insertion of the sheath necessarily requires an opening, or puncture wound, in the blood vessel so that a medical procedure can be performed through the sheath. After the medical procedure has been completed, the sheath must be removed from the blood vessel and the access hole in the blood vessel must be closed to create cessation of bleeding from the blood vessel.

As an alternative to the historically standard access to the cardiovasculature via the femoral artery in a patient's groin, access via an artery in a patient's wrist (i.e. either the radial artery or the ulnar artery) has gained recent popularity. This is particularly due to lessened post-procedure access site bleeding complications. The standard means for inducing post-procedure hemostasis of either a radial artery or an ulnar artery is to apply direct pressure to the patient's wrist approximate of the subcutaneous sheath entry site, or arteriotomy. Several devices have been introduced into the device market which aid in applying such direct pressure to a patient's wrist. These hemostasis devices are frequently composed of a wrist band with a means for focusing direct contact pressure on the patient's inside wrist skin surface approximate to the subcutaneous vessel's puncture wound. Such wrist band type devices may incorporate an inflatable balloon element for further focusing the direct pressure at the specific position on the patient's wrist.

A complication that can arise from compression type hemostasis devices is for the artery to collapse and become occluded, owing to the applied direct contact pressure. Such collapsing of a radial artery for instance, and the resulting non-patency, can create reduced blood flow to the patient's hand, as well as render the radial artery unusable for future percutaneous procedures. Arterial occlusion occurs in approximately 5-12% (see, e.g., 1, 2, 3 below] of patients undergoing procedures through the radial artery approach and therefore relates to a substantial patient population, particularly in high volume hospitals.
1. Agostoni P, Biondi-Zoccai G G, de Benedictis M L, et al. Radial versus femoral approach for percutaneous coronary diagnostic and interventional procedures. Systematic overview and meta-analysis of randomized trials. J Am Coll Cardiol. 2004; 44:349-356.
2. Bertrand O F, Rao S V, Pancholy S, et al. Transradial approach for coronary angiography and interventions: results of the first international transradial practice survey. J Am Coll Cardiol Interv. 2010; 3:1022-1031.
3. Rashid M, Kwok C S, Pancholy S, Chugh s, Kedev S A, Bernat I, Ratib K, Large A, Fraser D, Nolan J, Mamas M A. Radial artery occlusion after transradial intervntions: A systemic overview and meta-analysis. J Am Heart Assoc. 2016; 5:e002686 doi: 10.1161/JAHA.115.002686.

Embodiments of the present invention offer a means for facilitating hemostasis at a radial or ulnar artery puncture while avoiding the deleterious conditions that can result from arterial occlusion after administering direct pressure at the access site.

Description of the Related Art Section Disclaimer: To the extent that specific patents/publications/products are discussed above in this Background Section or elsewhere in this Application, these discussions should not be taken as an admission that the discussed patents/publications/products are prior art for patent law purposes. For example, some or all of the discussed patents/publications/products may not be sufficiently early in time, may not reflect subject matter developed early enough in time and/or may not be sufficiently enabling so as to amount to prior art for patent law purposes. To the extent that specific patents/publications/products are discussed above in this Background Section and/or throughout the application, the descriptions/disclosures of which are all hereby incorporated by reference into this document in their respective entirety(ies).

### SUMMARY OF THE INVENTION

It is therefore a principal object and advantage of the present invention to provide a tissue puncture closure assembly comprised of a closure device for insertion into and sealing of a blood vessel wall puncture that overcomes the shortcomings of direct overlying compression type closure systems described supra.

Another object and advantage of the present invention is the use of expansive force rather than compressive force against the wall of a blood vessel, i.e. force applied to the inner aspect of the artery to achieve arteriotomy closure.

It is a further an object and advantage of the present invention to provide a closure implant that rapidly and completely dissolves (biodegrades) in vivo, allowing for future arterial access, i.e. ‘re-sticks’.

In accordance with the foregoing object and advantages, an embodiment of the present invention provides a closure device that includes an absorbable anchor, or footplate, for insertion through the blood vessel wall puncture.

Embodiments of the footplate comprise a biocompatible and biocorrodible metal comprising a magnesium alloy, e.g. Mg1Al, Mg3Al, Mg6Al, Mg8Al (see, e.g., U.S. Pat. Nos. 9,155,530 and 9,456,816, and related descriptions of magnesium alloys and implants including footplates). Dissolution, or absorption, of magnesium alloy in vivo is an electrochemical corrosion process whereby blood, or bodily fluid, acts as an electrolyte and the Mg alloy implant, with its greatly negative electrochemical potential, acts as a ‘sacrificial anode’ in the resulting electrochemical cell formed at the implant's surface. As such, the dissolution of an absorbable magnesium alloy implant is a surface phenomenon. Therefore, the time to complete absorption of the footplate can be altered by adjusting the surface area-to -volume ratio. Further, the chemical constituency of the alloy can greatly influence the rate at which the implant, or footplate, dissolves, or absorbs. The footplate embodiments presented herein are configured to have a maximized surface area-to-volume ratio, and further are comprised to have a chemical constituency such that when presented inside the blood vessel, will completely dissolve in a period of hours, or preferably, shortly after the procedure and before the patient leaves the hospital.

The rate of dissolution of the magnesium alloy footplate can be further accelerated by surface modification, i.e. surface pretreatment. In brief, the process involves contacting the alloy with a specially prepared aqueous solution by dipping, spraying, or brushing followed by rinsing and drying in clean water. The solution is defined by the addition of a suitable acid to activate the alloy and modify the pH of the solution, and an accelerant, which is specifically selected to achieve increased corrosion. Through this process, the surface composition of the alloy is modified by (1) enriching it in impurities already contained in the alloy as the Mg component corrodes preferentially, and (2) depositing product(s) from solution that are associated with the acid and accelerant addition. Suitable inorganic acids include sulfuric, nitric, hydrochloric, and phosphoric and phosphoric. Acid concentrations may range from 1 mg to 10 g per liter of solution. Suitable organic acids include citric, tartaric, acetic, and oxalic. Suitable accelerants are generally soluble transition metal salts, typically though not exclusively of iron, manganese, and cobalt. Accelerant concentrations are typically much less than acid concentrations and range from 0.01 to 1 g per liter of solution. The contact time between solution and treated surface may be varied to further adjust corrosion rate. Contact times may range from 5 seconds to 10 minutes based on the chemistry of the pretreatment solution and the alloy. After pretreatment, surfaces are rinsed thoroughly with distilled or deionized water to halt the interaction between the pretreatment solution and the alloy. No further treatment of the surface is needed prior to use. An example of the process is as follows.

### Surface Pretreatment Example

Mg alloy samples are pretreated by immersion in an aqueous solution of 1 g of 98% sulfuric acid H₂SO₄ and 0.04 g of ferrous sulfate FeSO₄ in 10 mL of distilled water. Samples are treated in batches of 25 for a minimum of 90 seconds and no longer than 120 seconds. Samples are rinsed and dried in air after immersion in the pretreatment solution. At this stage the samples are complete and ready for use.

Apart from the benefits of rapid dissolution, magnesium implants have the added attribute of possessing antibacterial properties, i.e. properties that prevent implant-associated infection. In various studies [see, e.g., nos. 4, 5, 6 below], the proliferation of bacteria (e.g. Escherichia coli, Pseudomonas aeruginosa, Staphylococcus aureus) has been shown to be suppressed in the presence of metallic magnesium, similarly to the effects of bacterial antibiotics. These studies indicate that the antibacterial activity associated with metallic magnesium is largely attributable to an increase in pH (i.e. increase in alkalinity) at the implant site.
4. Rahim M I, Eifler R, Rais B, Mueller P P, Alkalization is responsible for antimicrobial effect of corroding magnesium. Journal of Biomedical Materials Research. 2015: 103, 11: 3526-3532, doi: 10.1002/jbm.a.35503
5. Yang L, Guangwang L, Zanjing Z, Lina L, Haowei L, et al. Antibacterial properties of magnesium in vitro and in an in vivo model of implant-associated methicillin-resistant staphylococcus aureus infection. Antimicrobial Agents and Chemotherapy. 2014: 58, 12: 7586-759,1doi: 10.1128/AAC.03936-14
6. Robinson D A, Griffith R W, Scechtman D, Evans R B, Conzemius M G, In vitro antibacterial properties of magnesium metal against Escherichia coli, Pseeudomonas aeruginosa and Staphylococcus aureus. Acta Biomaterialia. 2010: 6: 1869-1877, doi: 10.1016/j.actbio.2009.10.007

Embodiments of the footplate can be fabricated by using a number of manufacturing techniques. These include, but are not limited to, molding, extruding, machining, stamping, casting, forging, laser cutting and/or processing, laminating, adhesively fixing, welding, combinations thereof, among others, with effectiveness, as needed or desired.

In accordance with an embodiment of the present invention, the absorbable magnesium alloy footplate includes two thru-holes to allow passage of a filament, or suture. The filament loops through the two holes in the footplate and extends proximally to a position outside of the patient's body. The filament may be a continuous length and arranged as two substantially parallel legs. After a period of time elapses such that the footplate has substantially or fully dissolved, the filament is removed from the patient via a proximally applied pulling force to one of the two filament legs.

Embodiments of the filament may include either a bioabsorbable or non-bioabsorbable material, both types of which are commercially available for use as sutures. Bioabsorbable filaments may be comprised of various hydrolysis-dissolvable materials including Polyglycolic Acid polymer, Polyglactin copolymer, Poliglecaprone copolymer, Polydioxanone polymer, or Catgut. Non-bioabsorbable filaments may be comprised of such materials as Polypropylene, Nylon (polyamide), Polyester, PVDF, PTFE, ePTFE, silk, stainless steel, or nitinol. Further, embodiments of the filament may be of either a braided or monofilament construction.

In another embodiment of the present invention, an absorbable filament may be tied and knotted approximate to an absorbable magnesium alloy footplate with two thru-holes such that a single leg extends proximally from the footplate to a position outside of the patient's body. After a period of time passes such that the footplate is substantially absorbed, the absorbable filament is cut off just below the patient's skin surface and the portion of filament remaining in the patient absorbs in the subcutaneous tissue underlying the skin.

In another embodiment of the present invention, the absorbable magnesium alloy footplate includes one thru-hole which provides passage of a single non-absorbable filament. On the lumen side of the footplate, an absorbable magnesium alloy crimp sleeve is secured to the end of the filament thus providing a stop such that the filament remains attached, or tethered, to the footplate. At such time that both the crimp sleeve and the footplate have sufficiently dissolved inside the blood vessel, the filament is completely removed from the patient by applying a pulling force to the end of the filament that extends outside of the patient's body.

Another embodiment of the present invention provides an absorbable magnesium alloy footplate with a single hole, through which an absorbable or non-absorbable filament either partially or fully extends. At the interface between the hole and the filament, the relatively soft magnesium alloy is deformed such that the filament is trapped and resultantly attached to the footplate. In the embodiment that includes a non-absorbable filament, at such time that the footplate has sufficiently dissolved, thereby providing mechanical detachment of the filament from the footplate, the filament is completely removed from the footplate and the patient by applying a pulling force to the end of the filament that extends outside of the patient's body. In the embodiment that includes an absorbable filament, at such time that the footplate has sufficiently dissolved, the absorbable filament is cut just below the level of the skin and the remaining filament bioabsorbs in the subcutaneous tissue underlying the skin.

Another aspect of the present invention provides a method for making a reliable connection between the footplate and a filament. The method provides an absorbable magnesium alloy footplate with a hole formed by a piercing die, with a sharp-tipped conical mandrel. The resulting punctured hole geometry is ‘puckered’ at the surface of the footplate opposite from where the sharpened tip first contacted the footplate. A filament is introduced through the hole and the puckered surface is deformed, or crushed, back to a substantially flat surface, thus trapping the filament at the interface between the filament and the footplate's hole. The following describes a more detailed example of the footplate-to-filament connection method.

### Footplate-Filament Connection Example

The footplate is laid over a substrate comprised of a relatively low durometer elastic membrane (e.g. a thermoplastic elastomer) such that the footplate's bottom surface is in direct contact with the elastomeric substrate. Next, a cylindrical piercing mandrel with a sharp-pointed conical tip is positioned at a central location on the topside of the footplate. A high amplitude impact force is then applied to the mandrel such that the sharp-pointed tip penetrates through the full thickness of the footplate and into the elastic membrane. In the process of penetrating the relatively soft magnesium alloy, the mandrel creates both a hole and a conical deformation extending from the bottom surface (underside) of the footplate.

Alternatively, the underlying substrate may be a higher modulus material (e.g. tool steel) with a conical depression along with a small central hole, coincident with where the mandrel is positioned, i.e. such that footplate material is trapped and coined at the interface between the mandrel and the conical depression, thus creating both a cone-shaped protrusion and a thru-hole at the bottom surface of the footplate.

Next, the filament is introduced through the pierced thru-hole on the top surface (topside) of the footplate and then the footplate and filament are positioned upside-down such that the bottom surface of the footplate (with its protruding cone-shaped feature), and a short length of filament, are facing in an upward direction. Now, the topside of the footplate is positioned over a substrate that is of a higher elastic modulus than the magnesium alloy (e.g. steel) which supports the footplate/filament assembly. In the next process step, a flat-ended, hollow, right circular cylindrical mandrel (whose central hole is slightly larger than the diameter of the filament) is placed over the filament and the mandrel's flat end surface is positioned to be in contact with the raised, upwardly facing conical feature, i.e. at the puckered material location.

Finally, an impact force is applied to the cylindrical mandrel such that the conically displaced material is swaged (deformed) back to an essentially flat condition, thus trapping the filament securely in the footplate. This example constitutes an embodiment of the piercing die method described above.

At such time that the footplate has sufficiently dissolved, the filament is then freed from its formerly crushed fixation to the footplate and is easily removed from the patient by applying a pulling force to the end of the filament that extends outside of the patient's body.

The invention further provides a delivery assembly for presenting the footplate and filament into the blood vessel and articulating the footplate to a position substantially parallel to the longitudinal axis of the blood vessel. The delivery assembly is comprised of two concentric cannulae (a delivery sheath and a pusher tube) with the footplate and its proximally extending filament nested in the annulus between the two cannulae. In an alternating ‘push cycle’ and ‘pull cycle’, the concentric cannulae act together to slide-ably deliver the footplate to the inside of the blood vessel.

Embodiments of the cannulae may be comprised of biocompatible, appropriately flexible, kink resistant, low durometer polymers such as Pebax, Silicone, Nylon (polyamide), Polyurethane, PTFE, FEP, ETFE, HDPE, etc. Cannulae embodiments can be fabricated by using a number of manufacturing techniques. Those include, but are not limited to, molding, extruding, machining, laser cutting and/or processing, radio frequency (RF) forming and/or tipping, adhesively fixing, with effectiveness, as needed or desired.

The closure device is intended to be used in conjunction with a guidewire. A guidewire is a small diameter, flexible wire that is inserted percutaneously into the patient's blood vessel at the beginning of the procedure to act as a guide for navigating the medical instruments (e.g. catheters, etc.) used during the procedure and subsequent insertion of the stiffer and larger diameter shaft of the closure device. Guidewires for use in trans-radial procedures are typically in a range between 0.015 and 0.025 inches in diameter, i.e. relatively small diameter as compared to guidewires used in femoral access. The inner cannula, or pusher tube, provides a central passageway for a guidewire. The passageway facilitates insertion of the delivery assembly over a guidewire and through the percutaneous tissue tract and into the blood vessel. Once the delivery assembly has been introduced over the guidewire and the tapered distal margin of the delivery assembly is positioned inside the blood vessel, the guidewire is removed from the blood vessel and the delivery assembly by pulling the guidewire in a proximal direction, away from the patient.

In an embodiment of the present invention, the outer cannula, or delivery sheath, houses the pusher tube, footplate, and filament. The inside diameter of the delivery sheath is configured to be larger than the outside diameter of the pusher tube except at the tapered distal margin of the delivery sheath, where the delivery sheath is configured with a taper, i.e. a conical section that is smaller in diameter than the outside diameter of the pusher tube. In the default position, the pusher tube's distal tip resides just proximal of the taper in the delivery sheath's tapered distal margin. In order to provide passage of the pusher tube and footplate through the smaller diameter tapered distal margin of the delivery sheath, the delivery sheath includes a v-groove or partial skive at its top-dead-center position such that when the larger diameter pusher tube is motivated distally and comes into concentric contact with the delivery sheath's smaller inside diameter, the delivery sheath splits (tears open) locally at its tapered distal end to allow passage of the pusher tube and footplate.

Further, the pusher tube includes a vertical ledge feature which abuts the proximal end of the footplate such that when the pusher tube is motivated distally, the ledge feature applies a compressive force to the proximal end of the footplate. This distal displacement of the pusher tube (the push cycle) displaces both the pusher tube and the nested footplate from within the delivery sheath and into the blood vessel lumen. In the post-push cycle position, the footplate is fully displaced from within the delivery sheath, but remains approximated to the pusher tube and tethered to the delivery assembly via the filament leg, or legs. Immediately following the push cycle, the pusher tube is retracted in a proximal direction, i.e. the pull cycle. The pull cycle returns the pusher tube to its default position inside of the delivery sheath while simultaneously stripping the footplate from the pusher tube and leaving the footplate entirely distal of the delivery sheath, inside the arterial lumen. This stripping action occurs when the proximal end of the footplate comes into contact with the bottom distal tip of the delivery sheath. The proximal retraction of the pusher tube also aids to pull the filament leg, or legs, proximally owing to friction between the filament and the annulus formed between the outside surface of the pusher tube and the inside surface of the delivery sheath where the filament leg, or legs, reside.

Another aspect of the present invention provides an angled tip at the distal most margin of the delivery sheath. When the aforementioned frictional force is applied to the filament leg, or legs, the footplate is motivated in a proximal direction such that when it comes into contact with the angled distal end of the delivery sheath, the footplate articulates to an orientation substantially perpendicular to the longitudinal axis of the delivery sheath and thereby substantially parallel with the longitudinal axis of the blood vessel. This intentional articulation, or rotation, of the footplate aids in disallowing the footplate from inadvertently exiting the blood vessel once introduced and then, when the delivery assembly is subsequently retracted proximally, the footplate is positioned to reliably approximate against the wall of the blood vessel to effect hemostasis.

Another aspect of the present invention provides depth markings, or graduations on the outside surface of the delivery sheath to provide the user with feedback regarding how far the delivery assembly is inserted into the patient. For example, the graduations may be positioned at increments of one centimeter, thus indicating to the user how deeply the delivery assembly has been inserted below the level of the patient's skin and as a reference for the user to maintain the depth position after initial insertion and during the push and pull cycles, i.e. the deployment sequence. The graduations may be formed by transfer printing, or pad printing, of biocompatible indicia (ink) on the surface of the delivery sheath. The graduations may also be screen printed or inkjet printed on the surface of the delivery sheath. The process may also include a surface pretreatment process before the indicia is applied. An example of such a surface pretreatment is plasma pretreatment which increases the surface energy of the delivery sheath surface and improves wettability which translates to improved ink adhesion.

Another aspect of the present invention provides a radiopaque marking, or marker band, at or near the distal margin of the delivery sheath to allow the distal portion of the device to be positioned within the artery with the aid of fluoroscopy. Such a biocompatible, radiopaque 360 degree marker band provides the operator with additional feedback related to the proper positioning of the delivery assembly (within the arterial lumen) prior to deployment of the closure device. The marker band may be comprised of a biocompatible, highly loaded tungsten-filled thermoplastic polymer that may be heat fused to the surface of the delivery sheath, or alternatively, a biocompatible ink comprising radiopaque fine particles of materials such as platinum, tungsten, or barium sulfate.

Another aspect of the present invention provides a biocompatible hydrophilic coating that may be applied and bound to the surface of the delivery sheath. Such hydrophilic coatings absorb and bind water to the hydrophilic surface, i.e. induce dynamic hydrogen bonding with surrounding water. These chemical interactions with water give rise to hydrogel materials that exhibit extremely low coefficients of friction, thus greatly improving lubricity. As applied to the delivery sheath, such a hydrophilic coating improves device maneuverability and control, reduces localized tissue damage, and enhances patient comfort.

Another aspect of the present invention provides a method by which an operator may manipulate the delivery assembly after the footplate has been delivered inside the blood vessel lumen (as described supra). The method provides a deployment technique for seating the footplate, tensioning the filament leg, or legs, and securing the filament to provide temporary mechanical contact of the footplate with the blood vessel wall. The method includes the following steps.

Once the footplate has been successfully delivered inside the blood vessel lumen, the delivery assembly is retracted proximally such that the footplate is approximated against the blood vessel's inside wall. When the footplate is fully approximated (i.e. engaged with the vessel wall), the delivery assembly is pulled further proximally (away from the patient) such that the delivery assembly (delivery sheath and pusher tube together) fully exits the percutaneous tissue tract. This proximal motion, simultaneously applies continuous tension to the filament leg, or legs. Once the delivery assembly has been pulled proximally to the point where the filament leg, or legs, are exposed, proximal of the skin incision (outside of the patient's body), the user grasps the filament leg, or legs, and applies gentle tension, which maintains contact force of the footplate against the blood vessel wall, and hemostasis at the arteriotomy. Then, further pulling of the delivery assembly completely detaches the delivery assembly from the filament leg, or legs. The filament leg, or legs, having gentle tension applied to them, can be taped to the patient's arm in order to maintain tension on the filament legs and adequate contact force between the footplate and the artery wall, i.e. to maintain hemostasis.

The footplate is further aided in maintaining contact with the vessel wall by the patient's positive blood pressure that acts on the exposed surface of the footplate. The combination of the filament tethering the footplate and the positive blood pressure is sufficient to resist migration of the footplate during the time it takes for the footplate to dissolve. The filament legs remain taped to the patient's arm for a period of time such that the footplate has substantially dissolved within the artery.

At such time that the footplate has substantially dissolved, or absorbed, one of the two filament legs (for instance in the embodiment that includes two filament legs extending proximally) can be pulled in a proximal direction (away from the patient) while simultaneously holding pressure on the patient's skin such that the operator's forefinger and middle finger straddle the filament thus providing equal and opposite downward contact force on the skin and subcutaneous tissue while tension is applied to the filament during removal. This technique provides support of the subcutaneous tissue and underlying blood vessel wall, thereby lessening the likelihood that the filament removal will dislodge the thrombus plug at the arteriotomy. At this juncture, the filament has been completely removed from the patient and there exists a fully hemostatic condition at the puncture wound (arteriotomy) due to the natural coagulation of blood and resulting thrombus plug that is formed at the arteriotomy.

As described supra, in the embodiments that include a single, non-absorbable filament extending proximally, the single filament is pulled in a proximal direction in order to remove it from the patient. In the embodiments that include a single absorbable filament, the filament is intended to absorb in the subcutaneous tissue overlying the blood vessel. In such an absorbable filament embodiment, the portion of filament that extends proximally outside of the patient is cut just beneath the patient's skin and then disposed of

In another embodiment of the present invention, the taping of the filament leg, or legs, is replaced with a mechanical cinching device that comes into contact with the patient's skin and possesses adequate clamping force to maintain the necessary tension on the filament. One example of such a cinching device is commonly referred to as a “cord lock”.

In another embodiment of the present invention, the mechanical cinching device may be a smooth jawed ‘alligator clip’, clamped over the filament leg, or legs, at the skin surface with adequate clamping force to maintain the necessary tension on the filament and secure the footplate.

Another embodiment of the present invention provides a locking frame that is adhesively attached to the patient's wrist. The locking frame may be secured to the patient with a ‘peel and stick’ bottom surface that is positioned and adhered centrally to the percutaneous incision on the surface of the skin. Preferably, the peel and stick attachment of the locking frame is performed at the beginning of the procedure in order for the presence of blood on the patient's skin surface to not interfere with adhesion of the locking frame. The locking frame includes either one or two V-shaped locking elements, or ‘cleats’, that securely hold the filament leg, or legs, that extend outside of the patient's body. As an alternative to the adhesively attached locking frame, another possible embodiment provides a locking frame integrated into a band that is secured around the patient's wrist. Such a wrist band-type locking frame may be constructed with Velcro for purposes of securing the band to the patient's wrist. After a period of time has elapsed such that the footplate has substantially dissolved, the filament leg, or legs, are unlocked from the cleat, or cleats, and the filament is completely removed from the patient's wrist via a proximal pulling motion (away from the patient), and lastly, the locking frame is removed from the patient's wrist.

Another embodiment of the present invention provides a hemostatic pad that is incorporated with the filament locking means (e.g. an alligator clip) to aid in inducing hemostasis at the skin incision and the underlying tissue. The hemostatic pad is positioned to be in direct contact with the patient's skin, i.e. between the patient's skin and the filament locking device (e.g. an alligator clip) such that it is trapped to maintain firm contact against the skin. The pad is configured as a thin membrane with a partial slit and arranged such that the exposed portion of the filament (that which extends outside of the patient) passes through the slit. Hemostatic pads are typically comprised of a saturated gelatin sponge or felt-like material imbibed with one of three general categories of hemostatic agents, namely mechanical, active, and flowable [see, e.g., no. 7, below]. Mechanical hemostatic agents (e.g. porcine gelatin, cellulose, bovine collagen, or polysaccharide spheres) activate the extrinsic coagulation cascade and form a matrix at the bleeding site. Active hemostatic agents (e.g., bovine thrombin, recombinant thrombin, or pooled human plasma thrombin) stimulate fibrinogen at the bleeding site to produce a fibrin clot. Flowable hemostatic agents are composed of either a porcine or bovine gelatin matrix plus thrombin. These flowable hemostats provide both a mechanical and an active hemostat in a single product. Unlike mechanical hemostatic agents, active and flowable hemostatic agents do not require the normal hemostatic pathway as part of their mechanism of action and therefore continue to function even in the presence of anticoagulants like heparin, which is most frequently a necessary medicinal therapy in interventional cardiology procedures.
7. Schreiber M A, Neveleff D J, Achieving hemostasis with topical hemostats: making clinically and economically appropriate decisions in the surgical and trauma settings. AORN Journal. 2011: 94, 5: S1-S20, doi: 10.1016/j.aorn.2011.09.018

Another aspect of the invention provides a hand-held control assembly which provides improved ease of use and control in the actuation of the delivery assembly to facilitate the deployment motions described above, i.e. the push and pull cycles. The control assembly is comprised of a nested grasper and push button with an interposing bias member (coil compression spring) that allows the control assembly to alternate between a default configuration and a deployed configuration with a single push and release cycle whereby the user's grip posture is similar to that for the use of a syringe. The push button component is affixed to the proximal margin of the pusher tube and is slide-ably nested in the grasper handle which is affixed to the proximal margin of the delivery sheath. When the push button is depressed, it motivates the pusher tube in the distal direction, thereby motivating the footplate to extrude out of the delivery sheath in a distal direction, and into the blood vessel, thus the push cycle. Once the push button has been depressed, the push button is released. The releasing of the compressive force by the user's thumb on the push button allows the internal spring to reverse the direction of motion of the pusher tube (proximally) such that it reverts back to its default position within the delivery sheath, thus the pull cycle. This reverse (proximal) motion of the pusher tube further acts to pull the filament legs proximally (via friction, as described supra) which aids in positioning the footplate in an articulated orientation relative to the longitudinal axis of the delivery sheath, i.e. rotated and approximate to the angled distal tip of the delivery sheath, as previously described.

Embodiments of the control assembly components may be comprised of a large variety of engineering thermoplastics such as ABS, Polycarbonate, Nylon (polyamide), HDPE, PEEK, Polypropylene, PVC, etc. These components can be fabricated by using a number of manufacturing techniques. Those include, but are not limited to, injection molding, machining, or additive manufacturing techniques such as 3-D printing, SLA, SLS, with effectiveness, as needed or desired.

The push button component further includes a passageway for a guidewire to pass fully through its most proximal margin. The push button and the pusher tube (to which the push button is permanently affixed) provides a continuous channel through which a guidewire can slide-ably pass from inside the blood vessel lumen and internally through the closure device (from distal to proximal), and out of the proximal margin of the control assembly, outside of the patient's body. As described supra, once the closure device is introduced over the guidewire and into the patient's blood vessel, the guidewire is removed from the closure device by pulling it in a proximal direction (i.e. away from the patient), after which, the closure device is ready for actuation.

Another embodiment of the control assembly provides a hemostasis valve which is incorporated integrally with the guidewire passageway of the pusher tube and within the push button. The hemostasis valve disallows blood from passing from the arterial lumen, through the guidewire passageway, and out of the proximal end of the push button after the guidewire has been removed from the closure device, i.e. prior to deploying the closure device. The hemostasis valve is constructed as a discrete enclosure which houses a flexible dome-shaped diaphragm which is slit to accept passage of a guidewire. When the guidewire is removed (i.e. no longer present in the valve), the slit closes and seals the conduit to disallow blood flow through the guidewire passageway and out the proximal end of the closure device.

The control assembly may also include a feature, or features, that provide the user with positive tactile and/or audible feedback indicating that complete actuation of the push cycle has occurred. One embodiment of such a feature provides a metal tactile dome, or ‘snap dome’ positioned at the bottom of the grasper enclosure such that it is actuated (compressed and inverted) to provide a ‘click’ when the push button comes into contact with the snap dome, i.e. when the push button has reached its full stroke at the end of the push cycle. The audible click indicates to the user that the footplate has been fully displaced from within the delivery sheath and delivered inside the blood vessel lumen.

### BRIEF DESCRIPTION OF THE DRAWINGS

The present invention will be more fully understood and appreciated by reading the following Detailed Description in conjunction with the accompanying drawings, in which:

FIGS. 1a-1h are perspective views of footplates and filaments according to embodiments of the present invention.

FIGS. 2a-2d are perspective views of the footplate and filament connection method according to a preferred embodiment of the present invention.

FIG. 3a shows a perspective cut-away side view of the closure device in the default configuration, according to an embodiment of the present invention.

FIG. 3b is a close-up detail of the cut-away portion of FIG. 3a, according to an embodiment of the present invention.

FIG. 3c is a partially sectioned close-up perspective view of the distal portion of the closure device in the default configuration, showing the skive, or V-groove, in the top of the tapered distal margin of the delivery sheath, according to an embodiment of the present invention.

FIG. 4a is a perspective view of the closure device in the configuration at the end of the push cycle, according to an embodiment of the present invention.

FIG. 4b is a close-up detail of the distal end of the closure device shown in FIG. 3a, according to an embodiment of the present invention.

FIG. 5a is a perspective side view of the closure device in the configuration of a partially complete pull cycle, according to an embodiment of the present invention.

FIG. 5b is a close-up detail of the distal end of the closure device shown in FIG. 4a, according to an embodiment of the present invention.

FIG. 6a is a perspective view of the closure device in the configuration of a completed pull cycle, according to an embodiment of the present invention.

FIG. 6b is a close-up detail of the distal end of the closure device shown in FIG. 6a, according to an embodiment of the present invention.

FIG. 7 is a sectioned top view of the closure device in the default configuration with the control assembly sectioned, according to an embodiment of the present invention.

FIG. 8 is a sectioned top view of the closure device in the configuration of a completed push cycle with the control assembly sectioned, according to an embodiment of the present invention.

FIG. 9 is a sectioned top view of the closure device in the configuration of a completed pull cycle with the control assembly sectioned, according to an embodiment of the present invention.

FIG. 10a is a partially sectioned side view of the closure device, the blood vessel and tissue tract, and the user posture in the configuration of the initial step of the deployment sequence where the device is introduced over a guidewire, according to an embodiment of the present invention.

FIG. 10b is a close-up view of the distal portion of the closure device shown in FIG. 10a, according to an embodiment of the present invention.

FIG. 11a is a partially sectioned side view of the closure device, the blood vessel and tissue tract, and the user posture in the configuration of the step in the deployment sequence after the device has been introduced into the blood vessel and the guidewire has been removed, according to an embodiment of the present invention.

FIG. 11b is a close-up view of the distal portion of the closure device shown in FIG. 11a, according to an embodiment of the present invention.

FIG. 12a is a partially sectioned side view of the closure device, the blood vessel and tissue tract, and the user posture in the configuration of the deployment sequence where the push cycle has been completed, according to an embodiment of the present invention.

FIG. 12b is a close-up view of the distal portion of the closure device shown in FIG. 12a, according to an embodiment of the present invention.

FIG. 13a is a partially sectioned side view of the closure device, the blood vessel and tissue tract, and the user posture in the configuration of the deployment sequence where the pull cycle has been completed, according to an embodiment of the present invention.

FIG. 13b is a close-up view of the distal portion of the closure device shown in FIG. 13a, according to an embodiment of the present invention.

FIG. 14a is a partially sectioned side view of the closure device, the blood vessel and tissue tract, and the user posture in the configuration of the deployment sequence where the footplate is fully seated and the closure device deployment apparatus is partially removed from the patient, according to an embodiment of the present invention.

FIG. 14b is a close-up of the distal portion of the closure device shown in FIG. 14a, according to an embodiment of the present invention.

FIG. 15 is a partially sectioned side view of the closure device, the blood vessel and tissue tract, and the user posture in the configuration of the deployment sequence where the footplate is fully seated and the closure device deployment apparatus is completely removed from the patient, according to an embodiment of the present invention.

FIG. 16a-e are partially sectioned side views of various filament locking devices according to embodiments of the present invention.

FIG. 17 is a partially sectioned side view and a top view of a hemostatic pad positioned approximate to the patient's wrist for use in conjunction with a filament locking device (e.g. an alligator clip) according to an embodiment of the present invention.

FIG. 18 is a partially sectioned side view of the remaining portion of the closure device, the blood vessel and tissue tract, and the user posture in the configuration of the deployment sequence where the footplate has completely dissolved where all that remains of the closure device is the filament in the subcutaneous tissue tract and the temporary filament locking device (e.g. an alligator clip) which remains positioned at the skin surface, according to an embodiment of the present invention.

FIG. 19 is a partially sectioned side view of the remaining portion of the closure device, the blood vessel and tissue tract, and the user posture in the configuration after the footplate has completely dissolved and the operator has removed the temporary filament locking device (e.g. an alligator clip) and completely removes the filament from the patient by grasping and pulling the filament proximally, according to an embodiment of the present invention.

FIG. 20 is a sectioned side view of the blood vessel and tissue tract after the filament has been completely removed from the patient, rendering the arteriotomy fully hemostatic, according to an embodiment of the present invention.

### DETAILED DESCRIPTION

As mentioned earlier, vascular procedures are commonly performed through a puncture in either the radial artery or the ulnar artery. To close the puncture, often a compression device is utilized, which applies direct pressure to the skin surface on the inside of a patient's wrist, directed to compress the skin and subcutaneous tissue overlying the artery. These types of closure apparatus; however, can compress and collapse the arterial lumen, frequently rendering it non-patent. As an alternative to these ‘outside-in’ devices, the present invention describes a method and apparatus that creates hemostasis of an artery from the ‘inside-out’, i.e. a trans-radial or trans-ulnar closure device that applies expansive force rather than compressive force against the wall of a blood vessel.

The following detailed description contains certain references to positions identified as ‘distal’ and ‘proximal’. For clarity, these ‘distal’ and ‘proximal’ positions differ when referred to respective of; a) the closure device (the medical instrument), and; b) the patient.
a) When referring to the terms ‘distal’ and ‘proximal’ with respect to the closure device, distal is identified as the margin of the device that is forward of the user (closest to the patient), whereas proximal is identified as the margin of the device that is closer to the user, e.g. the control assembly is the most proximal portion of the closure device.
b) Quite oppositely, in cases where ‘distal’ and ‘proximal’ are referred to respective of positions on the patient, distal is identified as the position on the patient that is farther from the patient's heart, and proximal is identified as the position on the patient that is closer to the patient's heart, i.e. more cranial. By way of example, the tip of a patient's finger is more distal than the patient's wrist.

Reference will now be made in detail to the preferred embodiments of the present invention, examples of which are illustrated in the accompanying drawings.

In accordance with an embodiment of the present invention, the closure device 100, comprising a footplate 110 (the footplate may include any of the embodiments of the footplate , as discussed infra), and a filament 111 is provided and can be used to seal or close an opening formed through biological tissue, such as a percutaneously formed puncture (the puncture comprises the opening formed through the wall of the blood vessel and the tissue tract contiguous with the opening formed through the biological tissue, which extends through and to the skin overlying the blood vessel), an incision, or some other type of opening formed through biological tissue, such as a blood vessel, organ, or the like, to control (or prevent or stop) bleeding (or the flow of other biological fluid or tissue). For example, the closure device 100 of an embodiment of the present invention can be used to seal an arteriotomy 407, which is an opening, or incision, in an artery, such as the radial artery, and is formed in conjunction with a percutaneously formed puncture (an open tissue tract through the skin and tissue just above the blood vessel) by a clinician during a diagnostic or therapeutic intravascular surgical procedure.

In accordance with an embodiment of the present invention, and as elaborated in the subsequent descriptions, the closure device 100 may be in a pre-deployed configuration and position, or in a post-deployed configuration and position. A pre-deployed closure device configuration and position includes a configuration and position where the footplate 110 resides within the closure device 100. A post-deployed closure device configuration and position includes a configuration and position where the footplate 110 has been introduced through the arteriotomy 407 in the wall 123 of the blood vessel 121 and the footplate 110 resides inside the blood vessel 121 such that the footplate 110 is approximated against the blood vessel wall 123.

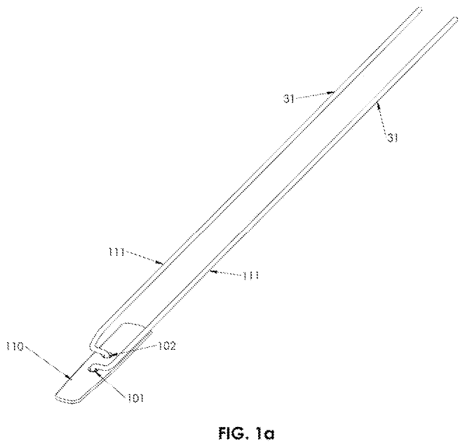

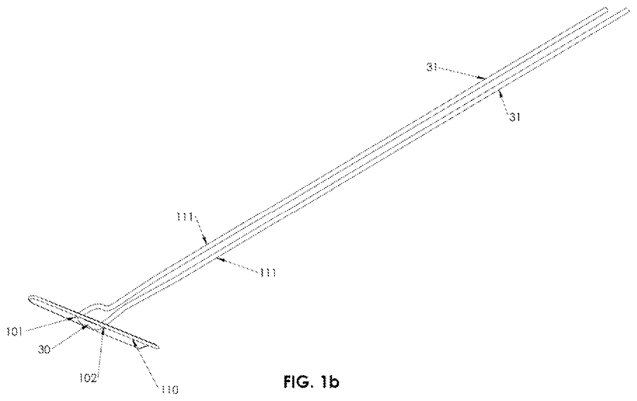

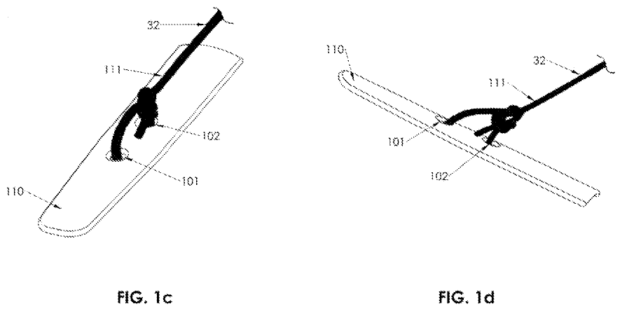

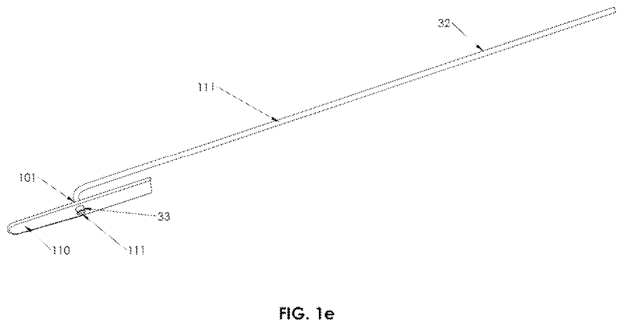

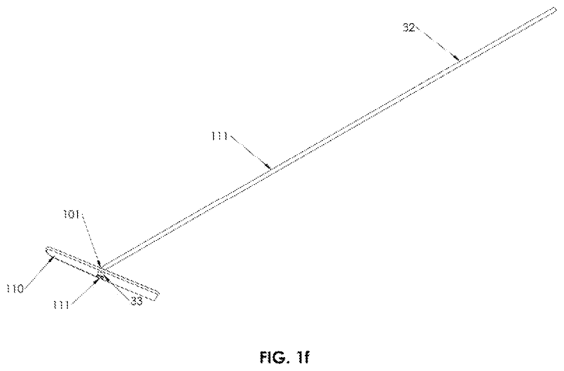

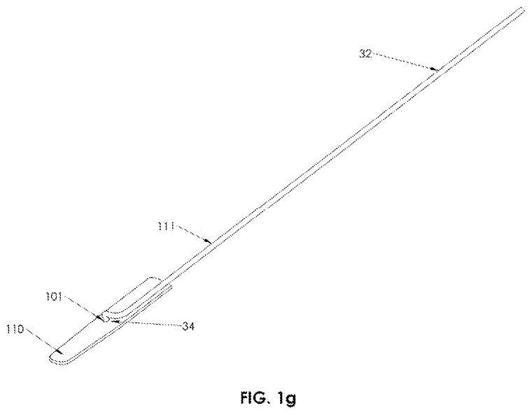

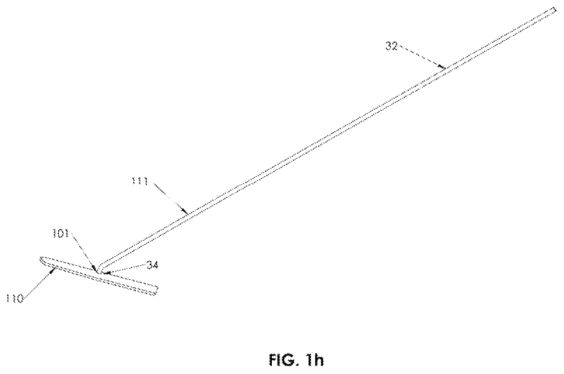

Referring now to the drawings in which like numbers refer to like parts throughout, FIG. 1a shows a footplate 110 according to an embodiment of the present invention. This embodiment shows a footplate 110 and a filament 111 in a pre-deployed closure device deployment configuration and position, wherein the footplate 110 is within the distal end of the delivery sheath 202 (not shown). The footplate 110 comprises a unitary flat or semi-flat plate (i.e. with an arcuate cross-section, as shown here and infra) with two thru-holes 101, 102, through which a filament 111 passes in a U-shaped loop 30 (hidden), whereby each of the two legs 31 of the filament 111 first extend laterally, adjacent to the bottom surface of the pusher tube 201 (not shown) and the top surface of the footplate 110, and then turn perpendicularly to extend proximally such that the two legs 31 are parallel with the longitudinal axis of both the footplate 110 and the pusher tube 201 (not shown).

Turning to FIG. 1b, the footplate 110 of FIG. la according to an embodiment of the present invention is illustrated. This embodiment shows the footplate 110 and filament 111 of FIG. la in a post-deployed closure device deployment configuration and position, wherein the footplate 110 is displaced outside of the distal end of the delivery sheath 202 (not shown) and resides within the lumen of the blood vessel 121 (not shown) in an orientation substantially perpendicular to the longitudinal axis of the closure device 100 (not shown). The footplate 110 comprises a unitary flat or semi-flat plate with two thru- holes 101, 102, through which a filament 111 passes in a U-shaped loop 30, whereby the filament 111 is parallel to the longitudinal axis of the closure device 100 (not shown) and has two longitudinally-extending legs 31.

Turning to FIG. 1c, the footplate 110 according to an embodiment of the present invention is illustrated. This embodiment shows the footplate 110 and filament 111 in a pre-deployed closure device configuration and position, wherein the footplate 110 is within the distal end of the delivery sheath 202 (not shown). The footplate 110 comprises a unitary flat or semi-flat plate with two thru-holes 101, 102, through which a filament 111 passes in a U-shaped loop 30 (not shown), whereby the filament 111 is tied and knotted approximate to the top surface of the footplate 110 and whereby the filament 111 is comprised of a bioabsorbable material and whereby the filament 111 is parallel to the longitudinal axis of the closure device 100 (not shown) and has a single longitudinally-extending leg 32.

Turning to FIG. 1d, a footplate 110 according to an embodiment of the present invention is illustrated. This embodiment shows the footplate 110 and filament 111 of FIG. 1c in a post-deployed closure device deployment configuration and position, wherein the footplate 110 is displaced outside of the distal end of the delivery sheath 202 (not shown) and resides within the lumen of the blood vessel 121 (not shown) in an orientation substantially perpendicular to the longitudinal axis of the closure device 100 (not shown). The footplate 110 comprises a unitary flat or semi-flat plate with two thru-holes 101, 102, through which a filament 111 passes in a U-shaped loop 30 (hidden) and whereby the filament 111 is tied and knotted approximate to the top surface of the footplate 110 and whereby the filament 111 is comprised of a bioabsorbable material and whereby the single, longitudinally-extending leg 32 is parallel to the longitudinal axis of the closure device 100 (not shown) and substantially perpendicular to the longitudinal axis of the footplate 110.

Turning to FIG. 1e, a footplate 110 according to an embodiment of the present invention is illustrated. This embodiment shows a footplate 110 and a filament 111 in a pre-deployed closure device deployment configuration and position, wherein the footplate 110 is within the distal end of the delivery sheath 202 (not shown). The footplate 110 comprises a unitary flat or semi-flat plate with one thru-hole 101, through which a filament 111 passes and is tethered to the footplate 110 by means of a crimp sleeve 33 that is crimped over the most distal end of the filament 111, and whereby the filament 111 is a single leg 32 which first extends laterally, adjacent to the bottom surface of the pusher tube 201 (not shown) and the top surface of the footplate 110. The filament 111 then turns perpendicularly to extend proximally such that it is parallel with the longitudinal axis of both the footplate 110 and the pusher tube 201 (not shown).

Turning to FIG. 1f, a footplate 110 according to an embodiment of the present invention is illustrated. This embodiment shows a footplate 110 and filament 111 of FIG. le in a post-deployed closure device deployment configuration and position, wherein the footplate 110 is displaced outside of the distal end of the delivery sheath 202 (not shown) and resides within the lumen of the blood vessel 121 (not shown) in an orientation substantially perpendicular to the longitudinal axis of the closure device 100 (not shown). The footplate 110 comprises a unitary flat or semi-flat plate with one thru-hole 101, through which a filament 111 passes and whereby the filament 111 is tethered to the footplate 110 by means of a crimp sleeve 33 that is crimped over the most distal end of the filament 111 and whereby the filament 111 is substantially perpendicular to the longitudinal axis of the footplate 110 and parallel to the longitudinal axis of the closure device 100 (not shown) and has a single longitudinally-extending leg 32.

Turning to FIG. 1g, a footplate 110 according to an embodiment of the present invention is illustrated. This embodiment shows a footplate 110 and a filament 111 in a pre-deployed closure device deployment configuration and position, wherein the footplate 110 is within the distal end of the delivery sheath 202 (not shown). The footplate 110 comprises a unitary flat or semi-flat plate with one thru-hole 101, through which a filament 111 passes and is affixed to the footplate 110 by means of a local deformation 34 of the footplate 110 at the region approximate to where the filament passes through the hole 101 of the footplate 110 and whereby the filament 111 is a single leg 32 which extends laterally, adjacent to the bottom surface of the pusher tube 201 (not shown) and the top surface of the footplate 110. The filament 111 then turns perpendicularly to extend proximally such that it is parallel with the longitudinal axis of both the footplate 110 and the pusher tube 201 (not shown).

Turning to FIG. 1h, a footplate 110 according to an embodiment of the present invention is illustrated. This embodiment shows a footplate 110 and filament 111 of FIG. lg in a post-deployed closure device deployment configuration and position, wherein the footplate 110 is displaced outside of the distal end of the delivery sheath 202 (not shown) and resides within the lumen of the blood vessel 121 (not shown) in an orientation substantially perpendicular to the longitudinal axis of the closure device 100 (not shown). The footplate 110 comprises a unitary flat or semi-flat plate with one thru-hole 101, through which a filament 111 passes and whereby the filament 111 is affixed to the footplate 110 by means of a local deformation of the footplate 110 at the region approximate to where the filament passes 34 and whereby the filament 111 is substantially perpendicular to the longitudinal axis of the footplate 110 and parallel to the longitudinal axis of the closure device 100 (not shown) and has a single longitudinally-extending leg 32.

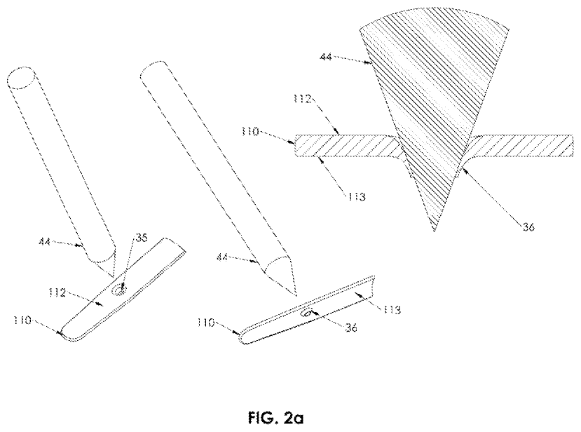

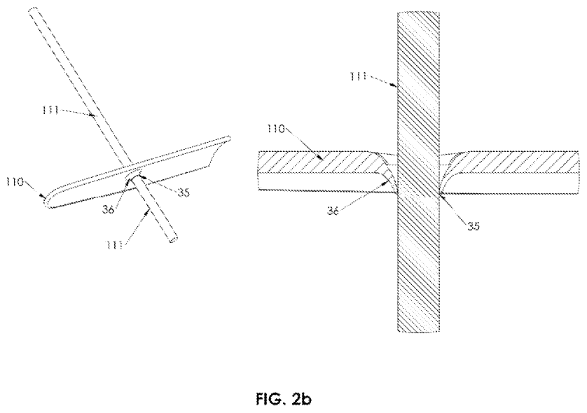

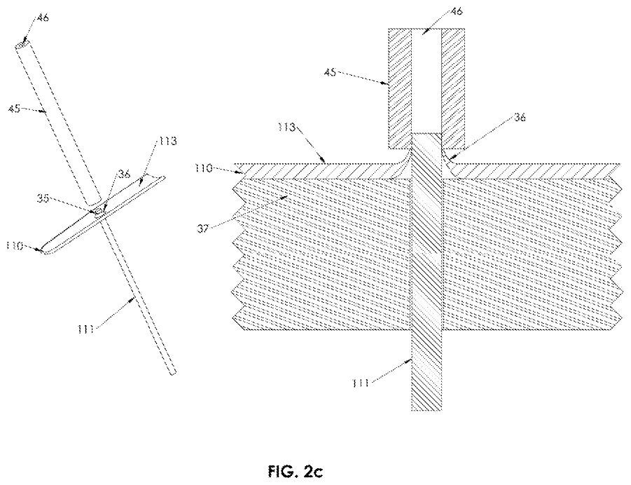

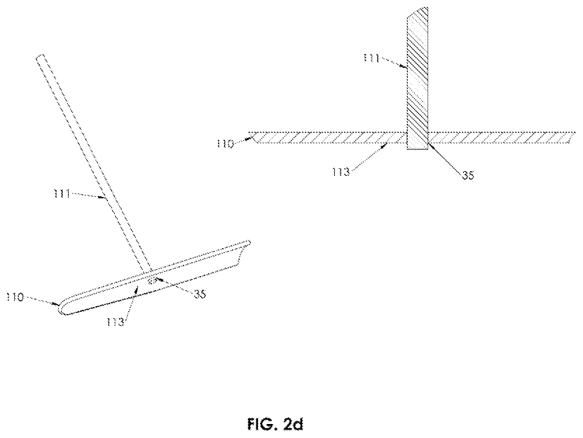

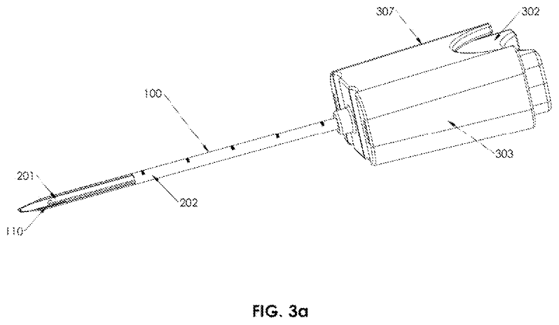

Turning to FIG. 2a, a footplate 110 according to an embodiment of the present invention is illustrated. This embodiment shows a footplate 110 whereby the footplate 110 comprises a unitary flat or semi-flat plate with a pierced thru-hole 35 whereby the pierced thru-hole 35 has been formed by a sharply tipped cylindrical piercing mandrel 44 that is introduced to the footplate 110 on the top surface of the footplate 112. The resulting geometry formed on (and through) the footplate 110 is that of a pierced thru-hole 35 surrounded by a dimpled, conically deformed feature 36, protruding from the bottom surface 113 of the footplate 110. Turning to FIG. 2b, a footplate 110 and a filament 111 according to an embodiment of the present invention is illustrated. This embodiment shows the footplate of FIG. 2a with a filament 111 passing through the pierced thru-hole 35 in the conically deformed feature 36, and extending in a substantially perpendicular orientation to the longitudinal axis of the footplate 110.

Turning to FIG. 2c, a footplate 110 and a filament 111 according to an embodiment of the present invention is illustrated. This embodiment shows the footplate 110 of FIGS. 2a and 2b with a filament 111 whereby the filament 111 passes through the pierced thru-hole 35 in the footplate 110. A flat-ended reforming mandrel 45 is positioned such that filament 111 passes through a central hole 46 in the flat-ended mandrel 45 and the flat-ended mandrel 45 is positioned approximate to the conically deformed feature 36 on the bottom surface of the footplate 110 and perpendicular to the longitudinal axis of the footplate 110. Further, the footplate 110 is shown positioned against a supporting plate 37. This positioning (collectively, the pre-reforming configuration) readies the footplate 110 and filament 111 for the impending reforming of the dimpled, conically deformed 36 feature on the bottom surface 113 of the footplate 110 described infra.

Turning to FIG. 2d, a footplate 110 and a filament 111 according to an embodiment of the present invention is illustrated. This embodiment shows the footplate of FIGS. 2a, 2b and 2c with a filament 111 whereby the filament 111 is affixed to the footplate 110 by means of swaging, i.e. a local deformation force applied to the bottom surface 113 of the footplate 110 approximate to the dimpled, conically deformed feature 36 (not shown) around the thru-hole 35 on the footplate 110 in FIG. 2c. The force is applied at the region approximate to where the filament 111 passes, such that the conically deformed feature 36 (not shown) is reconfigured to become substantially flat, thus trapping and affixing the filament 111 to the footplate 110.

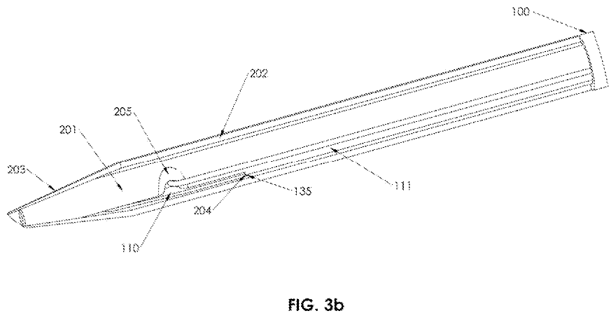

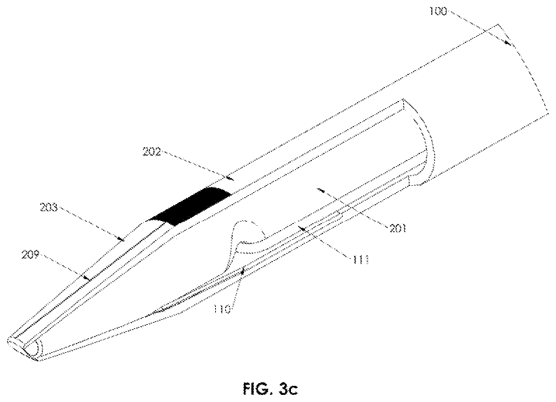

Turning to FIG. 3a, the closure device 100 according to an embodiment of the present invention is illustrated. This embodiment shows a perspective, cut-away (partially exposed) left side of the closure device 100. In accordance with an embodiment of the present invention, prior to insertion into a blood vessel 121 (not shown) that requires sealing (i.e. the default, or a pre-deployed closure device deployment configuration and position), the footplate 110 is positioned horizontally adjacent to the pusher tube 201 and nested between the pusher tube 201 and the delivery sheath 202. The pusher tube 201 is arranged to be concentric with the delivery sheath 202. At the proximal margin of the closure device 100 (i.e. at the control assembly 307), the pusher tube is attached to the push button 302 and the delivery sheath 202 is attached to the grasper 303, whereby the push button 302 is slide-ably nested within the grasper 303.

Turning to FIG. 3b, the closure device 100 according to an embodiment of the present invention is illustrated. This embodiment of the present invention shows a close-up perspective view of the distal portion of the closure device 100 shown in FIG. 3a, where the footplate 110, filament 111, and pusher tube 201 are shown in the default, or pre-deployed closure device deployment configuration and position and the pusher tube 201 fits concentrically inside the delivery sheath 202 and also where the delivery sheath 202 has a tapered distal margin 203. Also shown is a vertical ledge feature 204 on the pusher tube 201 that is in direct contact with the proximal end 135 of the footplate 110. The vertical ledge feature 204 on the pusher tube 201 provides engagement with the footplate 110 such that when the pusher tube 201 is motivated in a distal direction, it also pushes the footplate 110 in a distal direction to be delivered inside the lumen of the blood vessel 121 (not shown). Also shown is a radiused relief 205 on the pusher tube 201 which allows the filament 111 to nest and extend laterally at the interface between the top surface of the footplate 110 and the bottom surface of the pusher tube 201. Once the filament 111 has extended laterally, adjacent to the bottom surface of the pusher tube 201 and the top surface of the footplate 110, it turns perpendicularly to extend proximally such that it is parallel with the longitudinal axis of both the footplate 110 and the pusher tube 201.

Turning to FIG. 3c, the closure device 100 according to an embodiment of the present invention is illustrated. This embodiment of the present invention again shows a close-up perspective view of the distal portion of the closure device 100, where the footplate 110, filament 111, and pusher tube 201 are shown in the default, or pre-deployed closure device deployment configuration and position, and where the delivery sheath 202 has a tapered distal margin 203 such that the inside diameter of the tapered distal margin 203 of the delivery sheath 202 is smaller in diameter than the outside diameter of the pusher tube 201. This embodiment also shows where the delivery sheath 202 is provided with a partial skive, or V-groove 209 at the top of the tapered distal margin 203 of the delivery sheath 202 over the length of the tapered distal margin 203 of the delivery sheath 202. The V-groove 209 is provided to allow the delivery sheath 202 to split, or tear open, at the tapered distal margin 203 of the delivery sheath 202 when the pusher tube 201 is motivated distally to deliver both the distal portion of the pusher tube 201 and the footplate 110 out of the distal end of the delivery sheath 202 and into the lumen of the blood vessel 121 (not shown).

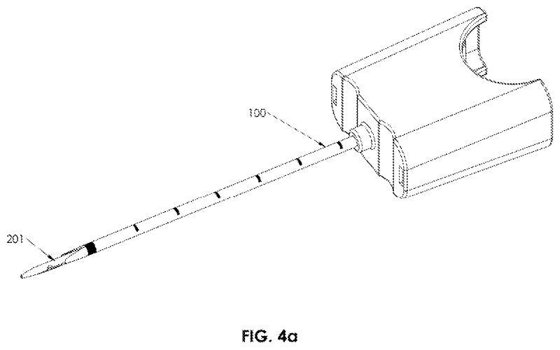

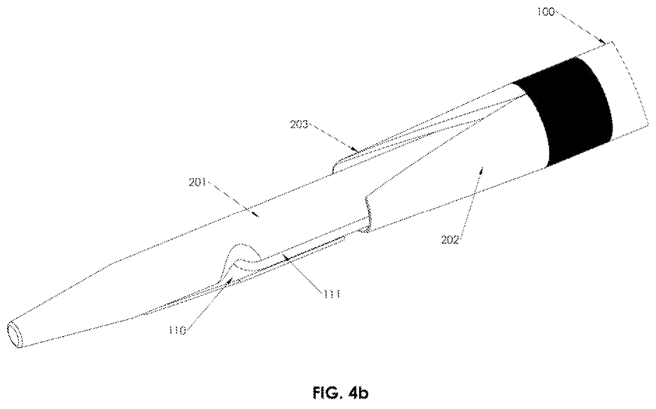

Turning to FIG. 4a, a perspective view of the closure device 100 according to an embodiment of the present invention is illustrated. This embodiment shows the left side of the closure device 100 in the configuration at the end of the push cycle after the closure device 100 has been inserted into a blood vessel 121 (not shown), i.e. a post-push cycle closure device deployment configuration and position where the push button 302 (hidden) has been depressed in a distal direction to motivate the pusher tube 201 in a distal direction.

Turning to FIG. 4b, a close-up view of the distal portion of the closure device 100, shown in FIG. 4a, in accordance with an embodiment of the present invention is illustrated. The embodiment shows the footplate 110, filament 111, pusher tube 201 and delivery sheath 202 in a post-push cycle closure device deployment configuration and position, i.e. after the closure device 100 has been inserted into the blood vessel 121 (not shown) and the delivery sheath 202 has split open at its tapered distal margin 203 such that the pusher tube 201 and the footplate 110 have been delivered out of the delivery sheath 202 via a user induced force being applied to the push button 302 (not shown) and into the blood vessel 121 (not shown).

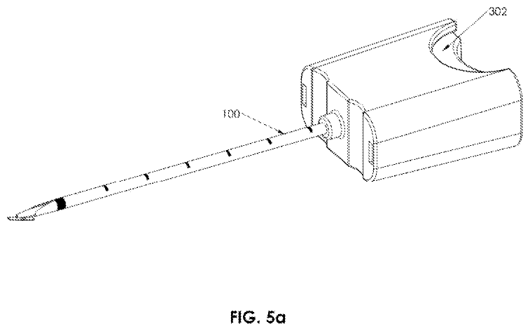

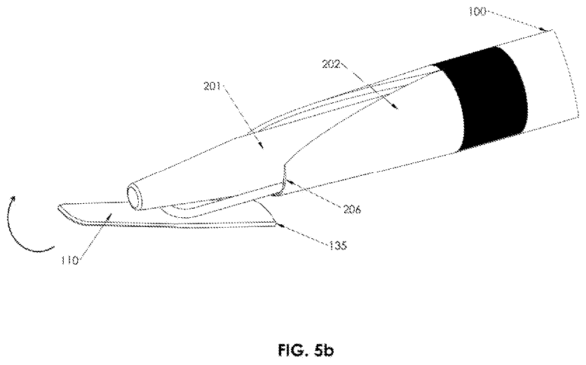

Turning to FIG. 5a, a perspective left side view of the closure device 100 according to an embodiment of the present invention is illustrated. This embodiment shows the closure device 100 in a partially completed pull cycle closure device deployment configuration and position where the push button 302 has been partially retracted via a proximally directed force provided by the bias member 304 (hidden).

Turning to FIG. 5b, a close-up view of the distal portion of the closure device 100, shown in FIG. 5a, in accordance with an embodiment of the present invention is illustrated. This embodiment shows the footplate 110, pusher tube 201, and the delivery sheath 202 in a partially completed pull cycle, i.e. after the pusher tube 201 has started to move in a proximal direction such that the footplate 110 begins to strip away from the pusher tube 201 as the proximal end 135 of the footplate 110 comes into proximity with the angled distal tip 206 of the delivery sheath 202 and the footplate 110 has started to articulate, or rotate, in a clockwise direction relative to the longitudinal axis of the delivery sheath 202, inside the blood vessel 121 (not shown).

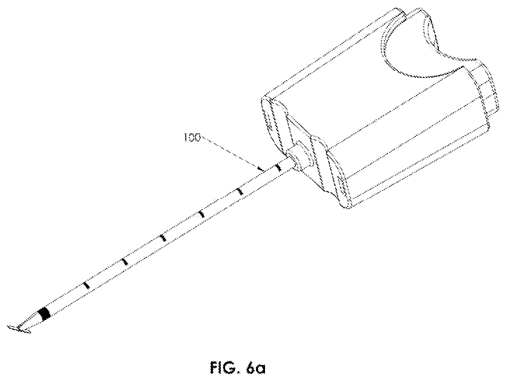

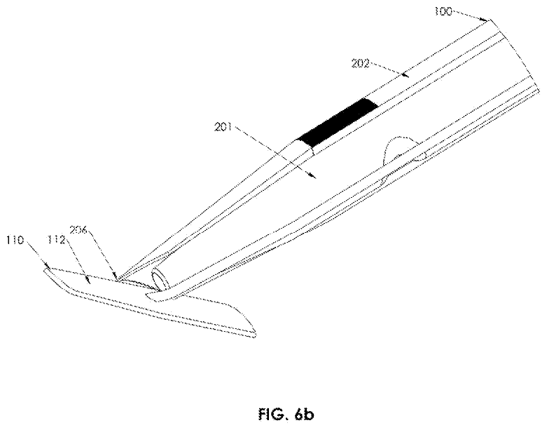

Turning to FIG. 6a, the closure device 100 according to an embodiment of the present invention is illustrated. This embodiment shows the left side of the closure device 100 at the end of the pull cycle after the closure device has been; a) inserted into the blood vessel 121 (not shown), and; b) the push cycle has been completed, and; c) the pull cycle has been completed, i.e. collectively the post-pull cycle closure device deployment configuration and position.

Turning to FIG. 6b, a close-up view of the distal portion of the closure device 100, shown in FIG. 6a, in accordance with an embodiment of the present invention is illustrated. This embodiment shows the footplate 110, the pusher tube 201, and the delivery sheath 202 in a fully completed, post-pull cycle closure device deployment configuration and position, i.e. after the pusher tube 201 has moved in a proximal direction such that the footplate 110 is completely stripped away from the pusher tube 201 and the top surface 112 of the footplate 110 comes into contact with the angled distal tip 206 of the delivery sheath 202 and the footplate 110 has fully articulated, or rotated, in a clockwise direction relative to the longitudinal axis of the delivery sheath 202, inside the blood vessel 121 (not shown), and positioned such that the longitudinal axis of the footplate 110 is substantially parallel with the vessel wall 123 (not shown), but prior to being fully seated (as is shown infra).

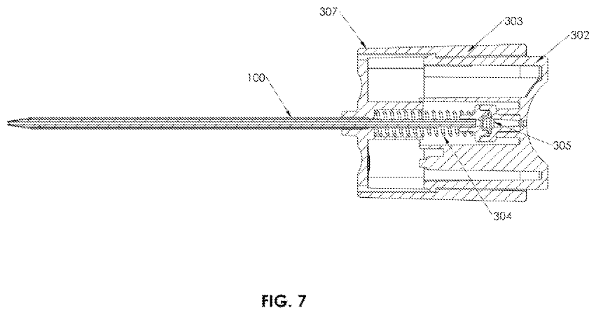

Turning to FIG. 7, a top section view of the closure device 100 according to an embodiment of the present invention is illustrated. This embodiment shows the closure device 100, including the proximal control assembly 307 (i.e. the grasper 303, the push button 302, the bias member 304, and the hemostasis valve 305; collectively the control assembly 307) in accordance with an embodiment of the present invention, prior to insertion into a blood vessel 121 (not shown) that requires sealing, i.e. the default, or a pre-deployed closure device deployment configuration and position.

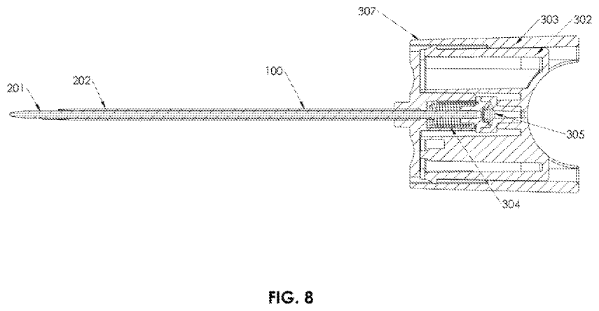

Turning to FIG. 8, a top section view of the closure device 100 according to an embodiment of the present invention is illustrated. The embodiment shows the closure device 100, including the proximal control assembly 307 (i.e. the grasper 303, the push button 302, the bias member 304, and the hemostasis valve 305; collectively the control assembly 307), the footplate 110 (hidden), the pusher tube 201, and delivery sheath 202 in a post-push cycle closure device deployment configuration and position, i.e. after the closure device 100 has been inserted into the blood vessel 121 (not shown) and the pusher tube 201 and the footplate 110 (hidden) have been delivered distally, out of the delivery sheath 202 (via a user induced distally directed force applied to the push button 302 and the resulting distal displacement of the push button 302 relative to the grasper 303) and into the blood vessel 121 (not shown) such that the delivery sheath 202 has split open to allow passage of the pusher tube 201 and the footplate 110 (hidden). The embodiment also shows the bias member 304 in a compressed configuration after the push button 302 has been displaced distally relative to the grasper 303.

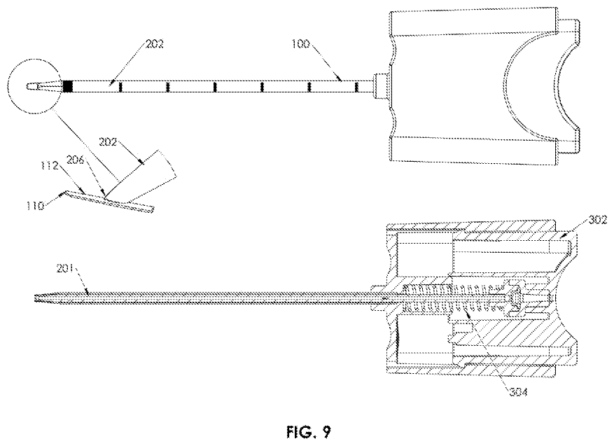

Turning to FIG. 9, both a top view and a top section view of the closure device 100 according to an embodiment of the present invention are illustrated. The embodiment shows the closure device 100 in a post-pull cycle closure device deployment configuration and position where the user has released the applied force on the push button 302 and the push button 302 and the pusher tube 201 retract in a proximal direction under the influence of the expansion of the formerly compressed bias member 304. This reverse (proximal) motion retracts the pusher tube 201 and the footplate 110 such that the top surface 112 of the footplate 110 is in direct contact with the angled distal tip 206 of the delivery sheath 202, as shown in a perspective close-up view. At this stage, the footplate 110 has fully articulated, or rotated, in a clockwise direction relative to the longitudinal axis of the delivery sheath 202, inside the blood vessel 121 (not shown), and is positioned such that the longitudinal axis of the footplate 110 is substantially parallel with the vessel wall 123 (not shown), but prior to being fully seated against the vessel wall 123 (not shown), as is illustrated and described infra.

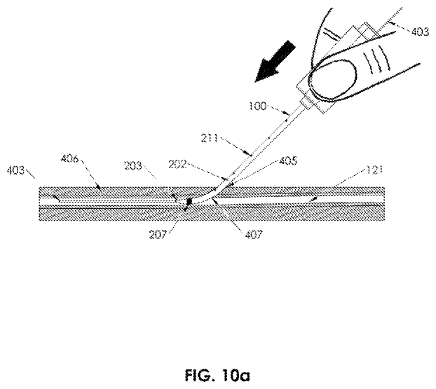

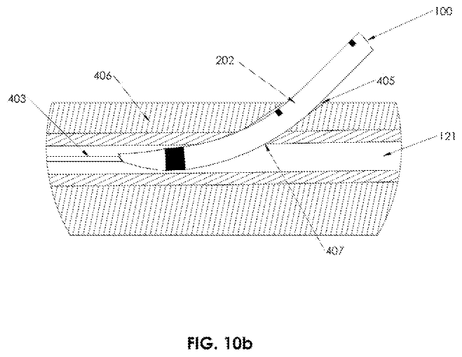

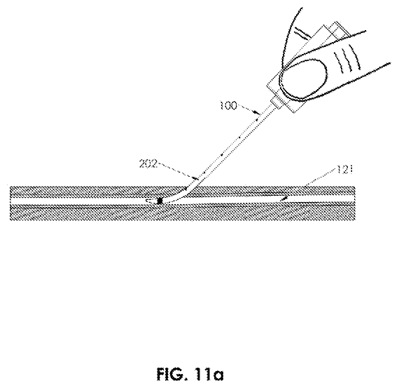

Turning to FIG. 10a, a partially sectioned left side view of the closure device 100, the blood vessel 121, and the user posture according to an embodiment of the present invention is illustrated. This embodiment shows the closure device deployment configuration and position where the closure device 100 has been inserted over a guidewire 403 such that the tapered distal margin 203 of the delivery sheath 202 is positioned inside the blood vessel 121, whereby the delivery sheath 202 has passed percutaneously through the skin incision 405, and farther, through the tissue tract in the subcutaneous tissue 406, and lastly, through the arteriotomy 407 and into the blood vessel 121. Also illustrated, according to an embodiment of the present invention, is a radiopaque marker band 207 on the distal portion of the delivery sheath 202 such that the distal portion of the delivery sheath 202 is positioned in the blood vessel 121 such that the radiopaque marker band 207 remains fully inside the blood vessel 121 and can be identified as such under fluoroscopic visualization, as necessary. The embodiment further shows a series of graduations, or markings 211, on the outside surface of the delivery sheath 202 thus indicating to the user how deeply the delivery sheath 202 has been inserted below the level of the skin incision 405 and as a reference for the user to maintain the depth position after initial insertion and during the push and pull cycles (i.e. the deployment sequence), as described infra.

Turning to FIG. 10b, a close-up view of the distal portion of the closure device 100 shown in FIG. 10a according to an embodiment of the present invention is illustrated. The embodiment shows the closure device 100 having been inserted over a guidewire 403, whereby the delivery sheath 202 has passed percutaneously in a distal direction through the skin incision 405, farther through the tissue tract in the subcutaneous tissue 406, and lastly, through the arteriotomy 407 and into the blood vessel 121.

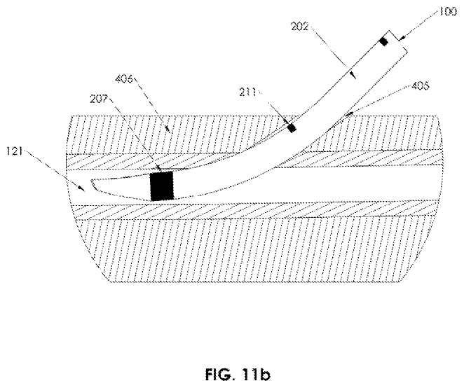

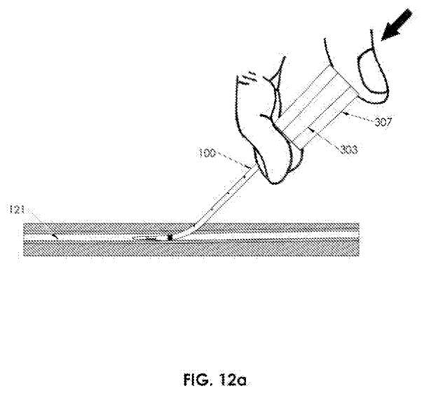

Turning to FIG. 11a, a partially sectioned left side view of the closure device 100, the blood vessel 121, and the user hand posture according to an embodiment of the present invention is illustrated. The embodiment shows the closure device 100 in the closure device deployment configuration and position where the closure device 100 has been inserted over a guidewire 403 (not shown) and the guidewire 403 (not shown) has been subsequently removed from the closure device and the blood vessel 121 by pulling the guidewire 403 (not shown) in a proximal direction, whereby the distal portion of the delivery sheath 202 is inside the blood vessel 121.

Turning to FIG. 11b, a close-up view of the distal portion of the closure device 100 shown in FIG lla according to an embodiment of the present invention is illustrated. The embodiment shows the closure device 100, the blood vessel 121, the percutaneous tissue tract in the subcutaneous tissue 406, and the skin incision 405. The distal portion of the delivery sheath 202 is positioned in the blood vessel 121 such that the radiopaque marker band 207 remains fully inside the blood vessel 121 and can be identified as such under fluoroscopic visualization, as necessary. The embodiment further shows a series of graduations, or markings 211, on the outside surface of the delivery sheath 202 thus indicating to the user how deeply the delivery sheath 202 has been inserted below the level of the skin incision 405 and as a reference for the user to maintain the depth position during the push and pull cycles (i.e. the deployment sequence), as described infra. In this configuration, the closure device 100 is ready for activation by the operator to deploy the system and deliver the footplate 110 (hidden) inside the blood vessel 121.

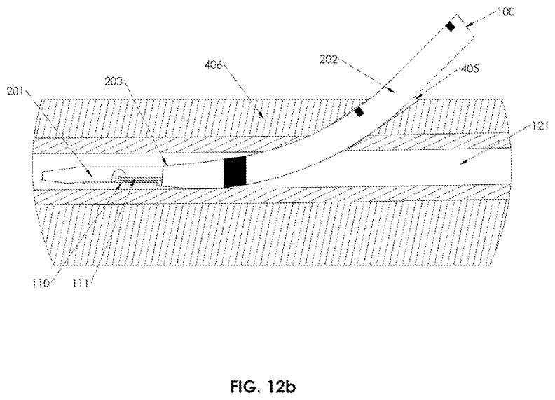

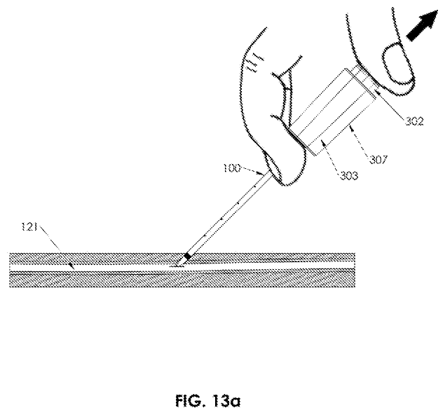

Turning to FIG. 12a, a partially sectioned left side view of the closure device 100, the blood vessel 121, and the user hand posture according to an embodiment of the present invention is illustrated. The embodiment shows the closure device 100 in a closure device deployment configuration and position where the closure device 100 has completed the push cycle, i.e. where the user has grasped the control assembly 307 and has fully depressed the push button 302 (hidden) relative to the grasper 303.

Turning to FIG. 12b, a close-up view of the distal portion of the closure device 100 shown in FIG. 12a according to an embodiment of the present invention is illustrated. The embodiment shows the closure device 100, the blood vessel 121, the percutaneous tissue tract in the subcutaneous tissue 406, and the skin incision 405 in a post-push cycle closure device deployment configuration and position. The embodiment further shows the footplate 110, the filament 111, the pusher tube 201, and the delivery sheath 202 after the closure device 100 has been inserted into the blood vessel 121 and the delivery sheath 202 has split open at its tapered distal margin 203 such that the pusher tube 201 and the footplate 110 have been delivered out of the delivery sheath 202 and into the blood vessel 121 via a user induced force being applied to the push button 302 (not shown).

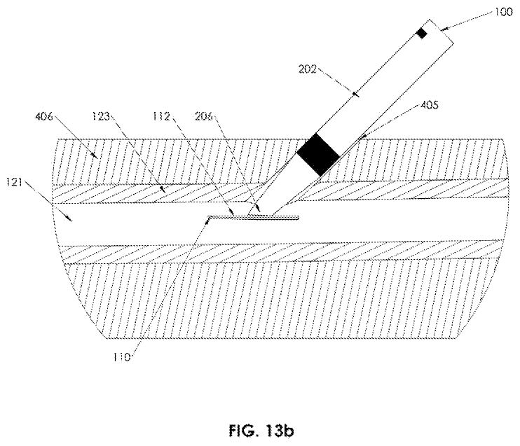

Turning to FIG. 13a, a partially sectioned left side view of the closure device 100, the blood vessel 121, and the user hand posture according to an embodiment of the present invention is illustrated. The embodiment shows the closure device 100 in a closure device deployment configuration and position where the closure device 100 has completed the pull cycle, i.e. where the user is grasping the control assembly 307 and has fully released the push button 302 relative to the grasper 303.

Turning to FIG. 13b, a close-up view of the distal portion of the closure device 100 shown in FIG. 13a according to an embodiment of the present invention is illustrated. The embodiment shows the closure device 100, the blood vessel 121, the percutaneous tissue tract in the subcutaneous tissue 406, and the skin incision 405 in a post-pull cycle closure device deployment configuration and position. The embodiment further shows the footplate 110 and the delivery sheath 202 in a fully completed pull cycle, i.e. after the pusher tube 201 (hidden) has moved in a proximal direction such that the footplate 110 is completely stripped away from the pusher tube 201 (hidden) and the top surface 112 of the footplate 110 has come into contact with the angled distal tip 206 of the delivery sheath 202 and the footplate 110 has fully articulated, or rotated, in a clockwise direction relative to the longitudinal axis of the delivery sheath 202, inside the blood vessel 121, and the footplate 110 is positioned such that the longitudinal axis of the footplate 110 is substantially parallel with the vessel wall 123, but prior to being fully seated against the vessel wall 123 (as is shown infra).

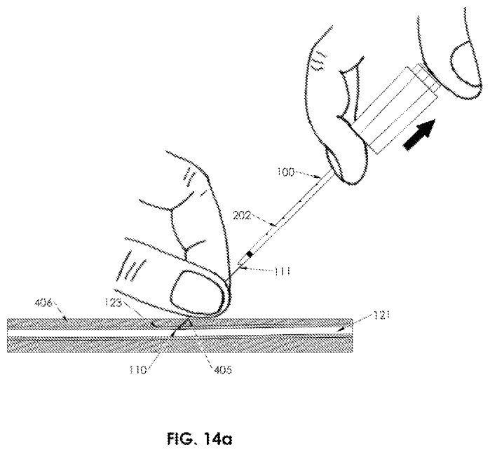

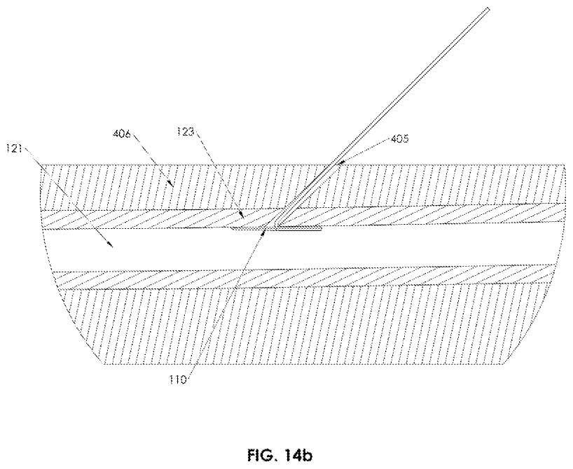

Turning to FIG. 14a, a partially sectioned left side view of the closure device 100, the blood vessel 121, and the user hand posture according to an embodiment of the present invention is illustrated. The embodiment shows the closure device 100 in a closure device deployment configuration and position where the closure device 100 has been retracted proximally such that the closure device 100 (including the delivery sheath 202) is fully outside of the percutaneous tissue tract in the subcutaneous tissue 406 and the skin incision 405, and simultaneously, the footplate 110 has come into complete approximation with the vessel wall 123. The closure device 100 is pulled proximally far enough so that the filament 111 is partially exposed such that the user grasps the filament 111 and applies gentle tension to the filament 111 to maintain contact of the footplate 110 with the vessel wall 123.

Turning to FIG. 14b, a close-up view of the distal portion of the closure device 100 shown in FIG. 14a according to an embodiment of the present invention is illustrated. The embodiment shows the blood vessel 121, the percutaneous tissue tract in the subcutaneous tissue 406, and the skin incision 405 in a closure device deployment configuration and position where the footplate 110 is fully seated (approximated) against the vessel wall 123.

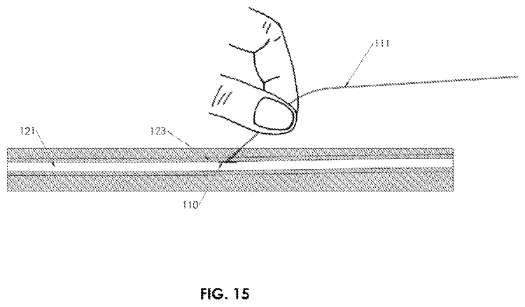

Turning to FIG. 15, a partially sectioned left side view of the blood vessel 121 with the footplate 110 fully approximated and the user hand posture according to an embodiment of the present invention is illustrated. The embodiment shows the closure device 100 (not shown) completely removed from the filament 111 (and the operative site) and the filament 111 being grasped by the operator in order to maintain gentle tension on the filament 111 to facilitate continued contact of the footplate 110 against the vessel wall 123, until a means of temporary securement can be applied to the filament 111 (as is shown infra).

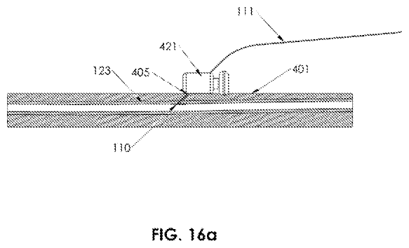

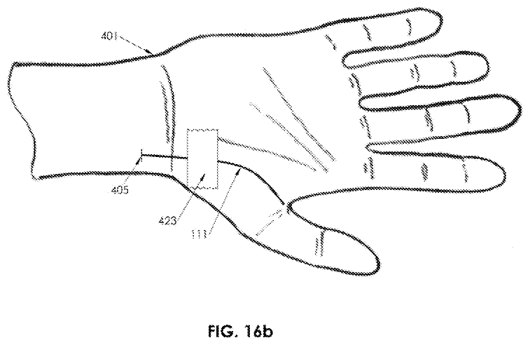

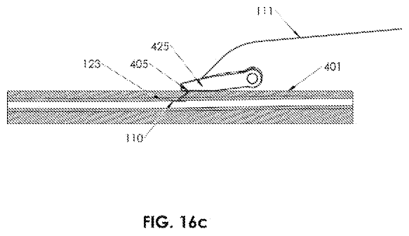

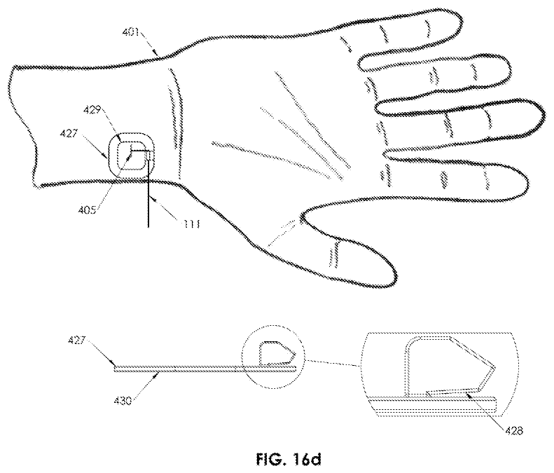

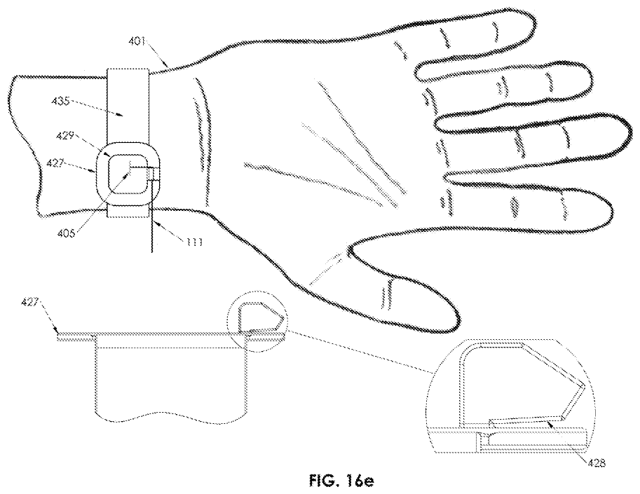

Turning to FIG. 16a shows a temporary filament locking device according to an embodiment of the present invention. In a partially sectioned left side view, the embodiment illustrates a cord lock 421 holding tension on the filament 111 approximate to the skin incision 405 on the patient's wrist 401 such that the tension on the filament 111 maintains approximation of the footplate 110 with the vessel wall 123 for a period of time such that the footplate 110 has substantially dissolved.

Turning to FIG. 16b shows a temporary filament locking means according to an embodiment of the present invention. In a top view, the embodiment illustrates an adhesive tape filament locking means 423 holding tension on the filament 111 approximate to the skin surface on the patient's wrist 401 and distal (on the patient) of the skin incision 405, or further distal on the patient (e.g. on the palm of the patient's hand), such that the tension on filament 111 maintains approximation of the footplate 110 (not shown) with the vessel wall 123 (not shown) for a period of time such that the footplate 110 (not shown) has substantially dissolved.

Turning to FIG. 16c shows a temporary filament locking device according to an embodiment of the present invention. In a partially sectioned left side view, the embodiment illustrates an alligator clip 425 holding tension on the filament 111 approximate to the skin incision 405 on the patient's wrist 401 such that the tension on the filament 111 maintains approximation of the footplate 110 with the vessel wall 123 for a period of time such that the footplate 110 has substantially dissolved.

Turning to FIG. 16d shows a temporary filament locking device according to an embodiment of the present invention. The embodiment illustrates a right side view of a filament locking frame 427 and a top view of the locking frame 427 attached to the patient's wrist 401 and holding tension on the filament 111. The locking frame has an opening 429 that when positioned and affixed to the skin surface of the patient's wrist 401, the opening 429 is central to the skin incision 405 such that it provides a sufficient opening for passage of the filament 111. The locking frame is provided with an adhesive peel-and-stick underside surface 430 which allows the locking frame 427 to be temporarily affixed to the wrist of the patient 401. The locking frame further provides a ‘cleat’ feature which is a V-groove 428 which holds the filament securely such that the tension on the filament 111 maintains approximation of the footplate 110 (not shown) with the vessel wall 123 (not shown) for a period of time such that the footplate 110 (not shown) has substantially dissolved.

Turning to FIG. 16e, shows a temporary filament locking device according to an embodiment of the present invention. The embodiment illustrates a right side view of a filament locking frame 427 and a top view of the locking frame 427 attached to the patient's wrist and holding tension on the filament 111. The locking frame has an opening 429 that when positioned and affixed to the patient's wrist 401, the opening 429 is central to the skin incision 405 such that it provides a sufficient opening for passage of the filament 111. The locking frame is provided with an integrated band 435 (similar to a wrist watch band) that is wrapped around the patient's wrist 401 and fastened (e.g. with Velcro or a buckle) to keep the locking frame 427 affixed to the patient's wrist 401. The locking frame further provides a ‘cleat’ feature which is a V-groove 428 which holds the filament securely such that the tension on the filament 111 maintains approximation of the footplate 110 (not shown) with the vessel wall 123 (not shown) for a period of time such that the footplate 110 (not shown) has substantially dissolved.

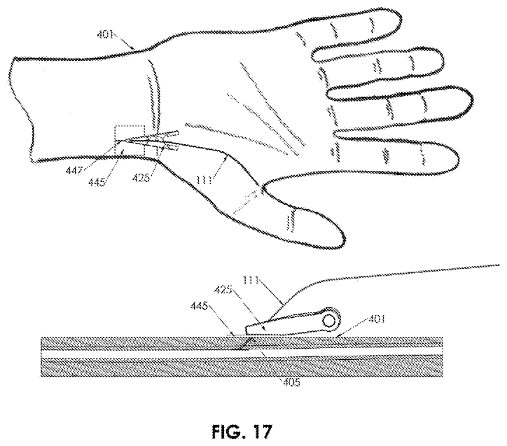

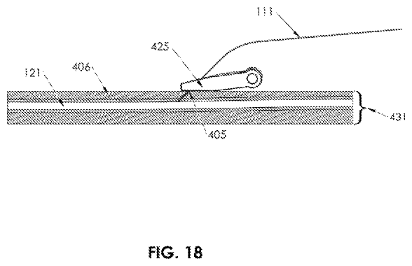

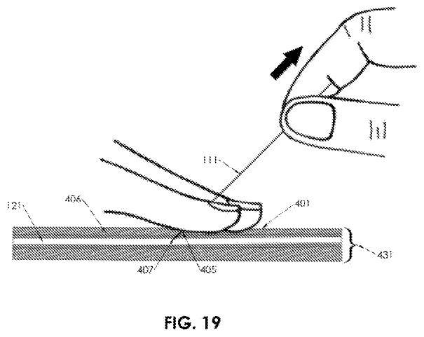

Turning to FIG. 17 shows a hemostatic pad 445 according to an embodiment of the present invention. In both a partially sectioned left side view and a top view showing the patient's hand and wrist 401, the hemostatic pad 445 is positioned such that it is in contact with the skin incision 405 on the patient's wrist 401, and further arranged such that the exposed portion of the filament 111 passes through a partial slit 447 in the hemostatic pad 445. The embodiment further illustrates the hemostatic pad 445 being sandwiched between a filament locking device (e.g. an alligator clip 425) and the skin incision 405 such that the hemostatic pad 445 is captured in firm contact against the skin of the patient's wrist 401. Turning to FIG. 18, a partially sectioned left side view of the blood vessel 121, the percutaneous tissue tract in the subcutaneous tissue 406, the skin incision 405, the filament 111, and the temporary filament locking device (e.g. an alligator clip 425) according to an embodiment of the present invention is illustrated. The embodiment shows the tissue layers 431 in a condition after a period of time has elapsed such that the footplate 110 (not shown) has fully dissolved in the blood vessel 121, i.e. the only remaining portion of the closure device 100 (not shown) is the filament 111 (in the percutaneous tissue tract in the subcutaneous tissue 406 and extending out of the skin incision 405) and the temporary filament locking device (e.g. an alligator clip 425), both of which will be removed from the patient (as is shown and described infra).

Turning to FIG. 19, a partially sectioned left side view of the blood vessel 121, the percutaneous tissue tract in the subcutaneous tissue 406, the skin incision 405, the filament 111, and the user posture according to an embodiment of the present invention is illustrated. The embodiment shows the tissue layers 431 in a condition after a period of time has elapsed such that the footplate 110 (not shown) has substantially dissolved, or absorbed, and the temporary filament locking device (e.g. an alligator clip 425, not shown) having been removed from the filament 111. The filament 111 is grasped by the operator and pulled in a proximal direction (away from the patient to completely remove the filament 111 from the patient) while simultaneously holding pressure on the skin surface of the patient's wrist 401 such that the operator's forefinger and middle finger straddle the filament thus providing equal and opposite downward contact force on the skin and subcutaneous tissue 406 (i.e. providing support and resistance to upward tissue displacement) while tension is applied to the filament during removal. At this juncture, the filament 111 is completely removed from the patient and there exists a fully hemostatic condition (i.e. complete cessation of bleeding) at the arteriotomy 407.

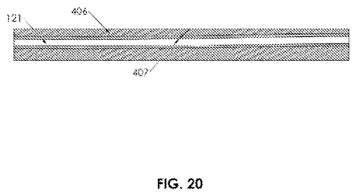

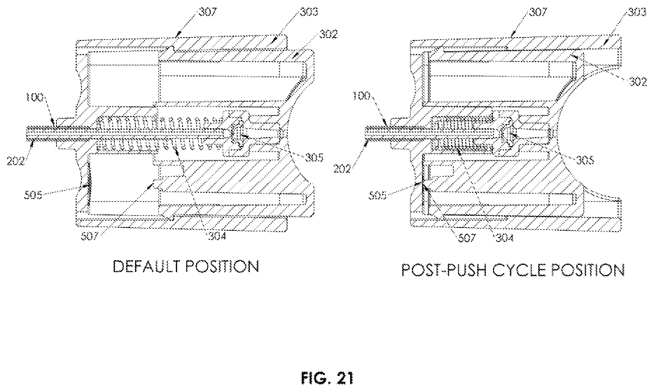

Turning to FIG. 20, a sectioned left side view of the blood vessel 121 and tissue tract in the subcutaneous tissue 406 after the filament 111 (not shown) has been completely removed from the patient, rendering the arteriotomy 407 in the blood vessel 121 fully hemostatic, according to an embodiment of the present invention.

Turning to FIG. 21, a sectioned top view of the control assembly 307 of the closure device 100 according to an embodiment of the present invention is illustrated. This embodiment shows the proximal control assembly 307 (i.e. the grasper 303, the push button 302, the bias member 304, and the hemostasis valve 305) of the closure device 100 in accordance with an embodiment of the present invention, prior to insertion into a blood vessel 121 (not shown) that requires sealing, i.e. the default, or a pre-deployed closure device deployment configuration and position. The embodiment further shows a metal snap dome 505 positioned at the bottom of the enclosure (inside distal margin) of the grasper 303 such that it is ready to be actuated (compressed and inverted) to provide a ‘click’ when a boss 507 on the distal margin of the push button 302 comes into contact with the snap dome 505, i.e. after the push button 302 is depressed by the user (relative to the grasper 303) and the push button 302 has reached its full stroke at the end of the push cycle. Also shown in another sectioned top view of the proximal control assembly 307 (i.e. the grasper 303, the push button 302, the bias member 304, and the hemostasis valve 305) of the closure device 100 is the post-push cycle closure device deployment configuration and position at the moment at which the snap dome 505 is being actuated to provide an audible ‘click’ when the boss 507 on the distal margin of the push button 302 contacts the snap dome 505, i.e. after the user-induced distally directed force is applied to the push button 302 and the resulting distal displacement of the push button 302 relative to the grasper 303 has occurred. The audible ‘click’ indicates to the user that the footplate 110 (not shown) has been fully displaced from within the delivery sheath 202 (partially shown) and delivered inside the lumen of the blood vessel 121 (not shown).

While embodiments of the present invention has been particularly shown and described with reference to certain exemplary embodiments, it will be understood by one skilled in the art that various changes in detail may be effected therein without departing from the spirit and scope of the invention supported by the written description and drawings. Further, where exemplary embodiments are described with reference to a certain number of elements it will be understood that the exemplary embodiments can be practiced utilizing either less than or more than the certain number of elements.

What is claimed is:

1. A closure device deployment device comprising:
a sliding member connected to the inside of a housing, and slidable within the housing between a first position and a second position in the distal direction along the longitudinal axis upon the application of a first force in the distal direction;
a sheath assembly extending along the longitudinal axis comprising a proximal end which is fixed to a distal end of housing;
a pusher tube extending along the longitudinal axis comprising a proximal end which is connected to a distal end of said sliding member and located concentrically within said sheath, wherein said pusher tube is movable along the longitudinal axis upon the application of the first force on said sliding member;
a filament extending along the longitudinal axis and located in an annulus between said sheath and said pusher tube;
a footplate connected to the distal end of said filament and extending along a plane that is parallel to the longitudinal axis, and structured to be within a distal end of said sheath assembly when the sliding member is in the first position and outside of the sheath when the sliding member is in the second position;
a bias member connected to the distal end of the sliding member such that when the sliding member is moved between the first position and the second position, the bias member is compressed and then when the force is released from the sliding member after the second position, the sliding member retracts proximally to a third position such that said pusher tube retracts proximally inside of said sheath, leaving said footplate outside of said sheath and in a position substantially perpendicular to the longitudinal axis.

2. The device of claim 1, wherein in the third position, a top surface of the footplate is in direct contact with the sheath assembly.

3. The device of claim 1, further comprising a radiopaque marker band around the distal end of the sheath assembly.

4. The device of claim 1, further comprising one or more markings, indicating depth, along the sheath assembly.

5. The device of claim 1, further comprising a passageway for a guidewire extending through the pusher tube.

6. The device of claim 5, wherein a hemostasis valve is positioned contiguous with the guidewire passageway and located at the proximal end of the housing.

7. The device of claim 1, wherein the pusher tube is tapered distally.

8. The device of claim 1, further comprising a longitudinal slit at the distal end of the sheath assembly.

9. The device of claim 1, further comprising a locking device tensioning the filament to maintain position of the footplate relative to the longitudinal axis in the third position.

10. The device of claim 9, wherein the locking device is an adhesive tape.

11. The device of claim 10, wherein the adhesive tape attaches a filament to an adjacent surface.

12. The device of claim 1, wherein in the third position, a portion of the filament is exposed and extends from the distal end of the sheath assembly.

13. The device of claim 1, further comprising an actuator on the housing connected to the bias member.

14. The device of claim 13, wherein when the actuator is depressed, the bias member is compressed and the sliding member is moved between the first position and second position.

15. The device of claim 14, wherein when the actuator is released after the sliding member is in the second position, the sliding member retracts proximally to the third position.
